(19) United States
(12) Patent Application Publication
(10) Pub. No.: US 2013/0071645 A1
(43) Pub. Date: Mar. 21, 2013

(54) METHOD FOR SEPARATING ATELOCOLLAGEN, METHOD FOR PREPARING MODIFIED ATELOCOLLAGEN, ATELOCOLLAGEN PREPARED BY USING THE SAME AND COLLAGEN-BASED MATRIX

(71) Applicants: Si-Nae Park, Seoul (KR); Sang-Hee Bae, Suwon-si Kyeonggi-do (KR); Yong Su Lee, Seoul (KR)

(72) Inventors: Si-Nae Park, Seoul (KR); Sang-Hee Bae, Suwon-si Kyeonggi-do (KR); Yong Su Lee, Seoul (KR)

(73) Assignee: DALIM TISSEN INC., Seoul (KR)

(21) Appl. No.: 13/698,017

(22) PCT Filed: Apr. 21, 2011
(86) PCT No.: PCT/KR2011/002903

§ 371 Date: Nov. 14, 2012

(30) Foreign Application Priority Data
May 14, 2010 (KR) 10-2010-0045322

(51) Int. Cl. C07K 1/34; B32B 9/02; B32B 37/16; C07K 14/78; C07K 1/30

(52) U.S. Cl. 428/318.6; 530/356; 156/307.3

(57) ABSTRACT

The present invention provides a method for separating an atelocollagen, wherein the process of ultrafiltration utilizing reusable filters and the process of diafiltration are combined, and by way of a single procedure line, atelocollagens can be extracted in a high purity from an animal tissue economically by removing impurities efficiently and conveniently.

### TECHNICAL FIELD

The present invention relates to a method for separating an atelocollagen and atelocollagens prepared in a high purity by using the same, particularly to a method for separating an atelocollagen, which comprises a step of: separating atelocollagen in a high purity from an animal tissue economically by removing impurities efficiently and conveniently, and atelocollagen prepared in a high purity by using the same.

In addition, the present invention relates to a method for preparing a modified atelocollagen that is dissolved in a neutral solution, outstanding in the biocompatibility, applicable for various formulations and convenient for use, and modified atelocollagens prepared in a high purity by using the same.

Further, the present invention relates to a collagen-based matrix that improves the mechanical strength and has a 3-dimensional matrix structure, and a method for preparing the same, which comprises steps of: forming a dense layer and a porous layer, and crosslinking them by using the above-mentioned atelocollagen and modified atelocollagens.

### BACKGROUND ART

In order to regenerate a biologically functional tissue, a culture environment capable of differentiating and proliferating various kinds of cells optimally should be established. For this purpose, extra-cellular matrix (ECM) plays a crucial role to maintain a tissue shape and support cell growth.

Basically, in order to create an artificial tissue, techniques for providing an environment of cell growth and proliferating cells are required. In histo-engineering fields for artificial tissue, extra-cellular matrix such as collagen or proteoglycan is being used to provide a milieu for cell growth, and further various growth factors are added for cell proliferation.

As biomaterials for artificial tissue, natural macromolecules are utilized widely and may include collagen, fibronectin, vitronectin, laminin and the like among extra-cellular matrices. For example, collagen or fibronectin contains a peptide sequence inducing cell adhesion and comprising arginine-glycine-aspartic acid (hereinafter, referred to as “RGD peptide”). RGD peptide can confer an environment causing cell adhesion on the surface of biomaterials, when being arranged artificially on this surface. Thus, it makes the surface of biomaterials bind with adjacent cells so as to mimic functions of an intrinsic tissue.

Especially, in biomaterial arts using proteins, collagen is believed most important among a variety of natural macromolecules as described above. The reason is that collagen is located in almost all tissues of a living body and makes a structural system for cell support and cell division. Further, collagen is an indispensable material in order to attach cells, form and maintain an organ and tissue, and finally construct a living body.

Collagen, a major protein component of extra-cellular matrix, exists very much in a hard tissue including bone and teeth, but also in a soft tissue including skin, tendon and blood vessel. In a mammal, it occupies approximately one third of whole proteins, and assembles cells sequentially so as to make a basic structure of a tissue or organ. Multi-cellular animal cannot survive without collagen. Therefore, the extra-cellular matrix of a tissue may confer a regenerative power of tissue cells on the lesion, whenever restoring toward a normal tissue in a living body. Hence, it is greatly advantageous to provide collagen for a basal matrix of artificial tissue substitutes.

There are a lot of tissues containing collagen such as skin, ligament, bone, blood vessel, amnion, heart sac, heart valve, placenta, cornea etc., but the kinds of collagen are different from one another, depending upon tissues. Especially, collagen type 1 is most widely used in histo-engineering fields, because it consists in almost all tissues including skin, ligament, bone etc.

In addition, collagen type 1 has a non-helical domain called telopeptide at both ends. This telopeptide becomes a major cause of immune reactions. Indeed, when collagen type 1 is used as raw material for medicine or cosmetics etc., atelocollagens are preferred by the removal of telopeptides.

Presently, general methods for separating collagen type 1 have adopted a procedure comprised of treating an animal tissue with trypsin, separating cells, then removing minerals and various proteins from extra-cellular matrix, and excluding other collagens insoluble in acids by using its intrinsic polarity or acid solubility. Typically, in the process for isolating collagen type 1, a precipitation, chromatography with a urea buffer, centrifugation and the like are being performed, and pepsin is treated to remove telopeptides causing an immune reaction as described above.

Unfortunately, there are a lot of problems in these methods. The process for separating collagen type 1 is inconvenient, and consumes time and cost a lot because of requiring a multi-step treatment. Further, in order to isolate collagen type 1 exclusively, requisite ureas should be removed in a later step, and pepsins should be removed or inactivated after removing telopeptides. That is to say, conventional methods for separating collagen type 1 make the isolation procedure more complicated and difficult. Either, they could make hard to economically obtain highly-purified collagen type 1.

Furthermore, biomaterial comprising collagen has a limitation due to the low strength and the biodegradable property. It cannot be applied directly to human tissues, even though collagen is effective to treat an injury.

Researches on artificial tissues using collagen have being actively advanced, aiming at such a property of collagen. Furthermore, methods for extracting collagen from animal tissues are being investigated.

Regarding to these, Korean Patent Publication No. 10-0465015 has described the method for preparing collagen type 1 in a high purity, which comprises steps of removing non-collagenous material by enzymatic treatment to lower immunity, extracting adipic impurities and insoluble collagen with an organic solvent, and removing them. However, in case of using an inorganic solvent as described in Korean Patent Publication No. 10-0465015, collagen may be denatured and a toxic organic solvent may cause an adverse action in a human, when applying for a human body,

In addition, in Korean Patent Publication No. 10-0676285, the method for isolating collagen from a pig, which comprises steps of reducing to powder or pieces with porcine bone tissues, cartilage tissues, skin tissues, tendon/sinew tissues, treating an acid, then treating pepsin repeatedly to isolate collagen type 1 primarily, repeating a salt treatment and titration toward a neutral condition 3 times to remove impurities, neutralizing at 30 to 37° C. of a low temperature, and then centrifuging to precipitate collagens, has been disclosed. The method of Korean Patent Publication No. 10-0676285 is advantageous to prevent collagen from being denatured and remove lipids or insoluble substances, but is complicated to comprise multi-steps of treating salts and titrating to a neutral state and inadequate to separate highly-purified collagen type 1 economically in a good yield.

Due to the disadvantages, it is believed that the process for separating collagen type 1 in a high yield from an animal tissue, while removing lipids, protein impurities, various insoluble impurities, residual urea, pepsin or other enzymes left behind, various salts and the like completely, is a very complex and difficult technique. In the meantime, in Korean Patent Publication No. 10-2002-0029859, the method for preparing an atelocollagen, which comprises steps of homogenizing a soft tissue or hard tissue of a mammal with an acid solution, then treating enzymes immediately, separating and purifying by a typical method, has been disclosed. Even if advantageously combining and simplifying foregoing steps before purifying collagen, this method has performed a dialysis and filtration in a later step of the purification so as to raise the purity.

Up to now, in the process for extracting collagen, several methods including dialysis, filtration repeated several times, or filtration by using overlapped 3 to 4 filters different in their pore sizes, have been adopted in order to increase the purity by removing various impurities after separating collagen primarily. However, the dialysis method cannot increase the purity sufficiently, because the pore size of a membrane is restricted between 12,000 to 14,000 Dalton. The filtration by using overlapped 3 to 4 filters different in their pore sizes cannot to be recycled and costs high, since it consumes several expensive filters.

Therefore, in this arts belonging to the present invention, it is necessary to provide a method for separating an atelocollagen, which comprises a step of: separating atelocollagen economically from animal tissues in a high purity by removing impurities efficiently and conveniently.

Accordingly, in order to settle foregoing problems of conventional methods as described above, the present inventors have provided a method for separating collagen, wherein an ultrafiltration using reusable filters and diafiltration are combined, and as a result, completed a method for extracting collagen in a high purity by way of a single procedure.

Also, the present inventors have developed a method for preparing modified atelocollagens that are dissolved in a neutral solution, outstanding in the biocompatibility, applicable for various formulations and more convenient for use. This method enables inherent collagens to be utilized directly for various fields including cosmetics, medicines and food etc. without any hydrolysis, including peptidosis and enzymatic digestion of collagens. Moreover, the present inventors have introduced a method for manufacturing a porous matrix to the above-mentioned method, which comprises steps of: forming a collagen bilayer structure comprising a dense layer and a porous layer (2 collagen layers different from each other in the porosity), and crosslinking them. Indeed, we have manufactured a 3-dimensional matrix structure that improves the mechanical strength, and as a consequence, solved problems in respect of the low strength and the biodegradable property.

### SUMMARY OF INVENTION

The object of the present invention is to provide a method for separating an atelocollagen, which comprises a step of: separating atelocollagen in a high purity from animal tissues economically by removing impurities efficiently and conveniently, and atelocollagen prepared in a high purity by using the same.

The another object of the present invention is to provide a method for preparing a modified atelocollagen that makes collagen dissolved even in a neutral solution to ameliorate the biocompatibility, being applicable for various formulations and more convenient for use, and modified atelocollagens prepared by using the same.

The another object of the present invention is to provide a collagen-based matrix that increases the mechanical strength and has a 3-dimensional matrical structure, and a method for preparing the same, which comprises steps of: forming a dense layer and a porous layer (2 collagen layers different from each other in the porosity), and crosslinking them by using the above-mentioned atelocollagen and modified atelocollagens.

### DETAILED DESCRIPTION OF INVENTION

Above all, terms used in this specification will be explained clearly as follows. As used herein, the term “ultrafiltration” refers to a method intervening between a precision filtration and reverse osmotic pressure method, particularly a method for separating and filtrating material by using a pressure gap caused over a filter membrane, in order to remove small molecules out of macromolecules. The filter membrane used in this ultrafiltration has a wide range of a pore size, depending upon target material.

As used herein, the term “diafiltration” refers to a method for gradually raising a ratio of desired material. During a diafiltration, among feeds (supply), retentates, and filtrates larger molecules than pores on a filter membrane are concentrated and smaller molecules are filtrated to pass through the filter membrane. But, among retentates, purified molecules are left partially and if desired, may be restored into feeds. In this case, the resulting molecules are diluted by adding purified water, concentrated repeatedly over a filter membrane and purified to gradually increase the portion of desired material.

Hereinafter, the present invention will be described clearly by using the above-mentioned terms used in this specification.

According to one aspect of the present invention, the method for separating an atelocollagen, which comprises steps of:

(a) preparing a sample containing atelocollagens without any telopeptide in a container;

(b) transferring the sample containing atelocollagens from the container toward a filtration module possessing a filter membrane, and performing an ultrafiltration by passing the sample through the filter membrane with applying a pressure on the filtration module to be filtrated;

(c) collecting an atelocollagen solution flowing out of the filtration module after filtrating over the filter membrane in the step of performing the ultrafiltration;

(d) measuring a flow rate of the atelocollagen solution filtrated over the filter membrane to determine an ultrafiltration rate;

(e) stopping the ultrafiltration when the ultrafiltration rate reaches a predetermined level or less;

(f) collecting a retentate of the sample excluded on the filter membrane and restored from the container, adding water to the collected retentate, and then transferring it to the filtration module possessing a filter membrane to perform a diafiltration;

(g) collecting an atelocollagen solution flowing out of the filtration module after filtrating over the filter membrane in the step of performing the diafiltration; and

(h) repeating the steps (f) and (g), is provided.

In the method for separating an atelocollagen according to one embodiment of the present invention, in the step (b), the sample containing atelocollagens is transferred from the container toward the filtration module possessing a filter membrane by pumping with a pump device, and about 10 to 30 psi of pressure is applied on the filtration module.

In the method for separating an atelocollagen according to one embodiment of the present invention, in the step (e), the ultrafiltration is stopped when the ultrafiltration rate decreasingly reaches about 1 g/min or less.

In the method for separating an atelocollagen according to one embodiment of the present invention, in the step (f), the same volume of purified water as that of the solution filtrated by the ultrafiltration may be added to the retentate restored to the container.

In the method for separating an atelocollagen according to one embodiment of the present invention, the diafiltration may be repeated at least 5 times.

In the meantime, the atelocollagen according to one embodiment of the present invention is prepared by the method for separating atelocollagen as described above.

According to another aspect of the present invention, the method for preparing a succinylated atelocollagen, which comprises steps of:

(a) reacting an atelocollagen solution with succinic anhydride, and maintaining the reaction solution of atelocollagen and succinic anhydride under a basic condition;

(b) stirring the reactant of atelocollagen and succinic anhydride for a predetermined period at a low temperature;

(c) maintaining the reactant of atelocollagen and succinic anhydride for a predetermined period at around pH of 9 to 10 after stirring in the step (b);

(d) converting the reactant of atelocollagen and succinic anhydride to an acidic state by adding acids, and forming a precipitate of succinylated atelocollagen;

(e) separating and obtaining the precipitate of succinylated atelocollagen, is provided.

In the method for preparing a succinylated atelocollagen according to one embodiment of the present invention, preferably the step (b) and the step (c) are repeated, and more preferably, the step (b) and the step (c) are repeated at least 4 times.

The method for preparing a succinylated atelocollagen according to one embodiment of the present invention further comprises a step of washing the precipitate of succinylated atelocollagen by using acidic distilled water. Preferably, the method for preparing a succinylated atelocollagen according to one embodiment of the present invention further comprises a step of lyophilizing the precipitate of the succinylated atelocollagen.

In the meantime, the succinylated atelocollagen according to one embodiment of the present invention is prepared by the method for preparing a succinylated atelocollagen as described above.

According to another aspect of the present invention, the method for preparing an esterified atelocollagen, which comprises steps of:

(a) preparing an atelocollagen colloid by adding atelocollagen in ethanol or methanol, converting the atelocollagen colloid to an acidic state by adding acids, and then stirring it;

(b) converting the atelocollagen colloid to a nearly neutral condition, after being stirred in the step (a);

(c) collecting a precipitate of esterified atelocollagen by centrifuging the atelocollagen colloid after being converted to a nearly neutral condition in the step (b); and

(d) pouring the precipitate of esterified atelocollagen obtained in the step (c) into a dialysis membrane to perform a dialysis in purified water, is provided.

Preferably, the method for preparing an esterified atelocollagen according to one embodiment of the present invention further comprises a step of lyophilizing the precipitate of esterified atelocollagen after being dialyzed in the step (d).

In the meantime, the esterified atelocollagen according to one embodiment of the present invention is prepared by the method for preparing an esterified atelocollagen as described above.

According to another aspect of the present invention, the method for manufacturing a collagen-based matrix, which comprises steps of:

(a) spreading uniformly the atelocollagen colloid obtained by the method for preparing an atelocollagen as described above to form a membrane with a uniform thickness, and then lyophilizing to form a porous layer of collagen;

(b) spreading uniformly the atelocollagen colloid obtained by the method for preparing an atelocollagen as described above, and pressing it with a porous adsorption plate to leach out water and closely attach collagen particles to form a dense layer of collagen;

(c) layering the porous layer of collagen formed in the step (a) on the dense layer of collagen formed in the step (b), and air-drying them so as to primarily bind the porous layer of collagen to the dense layer of collagen; and

(d) generating crosslinking between the porous layer of collagen and the dense layer of collagen bound primarily in the step (c) by using a crosslinking means so as to secondarily bind the porous layer of collagen to the dense layer of collagen, is provided.

The method for manufacturing a collagen-based matrix according to one embodiment of the present invention further comprises a step (e) of washing out a crosslinking agent, when the crosslinking means is a crosslinking agent.

More preferably, in the step (e), a bilayer comprising the dense layer of collagen and the porous layer of collagen is further lyophilized after washing out a crosslinking agent.

In the method for manufacturing a collagen-based matrix according to one embodiment of the present invention, preferably the pressure applied by the porous adsorption plate in the step (b) may be 1 to 20 psi.

In the method for manufacturing a collagen-based matrix according to one embodiment of the present invention, the crosslinking means may be EDC[1-ethyl-3-(3-dimethyl aminopropyl)carbodiimide] or glutaraldehyde.

In the method for manufacturing a collagen-based matrix according to one embodiment of the present invention, hyaluronic acid is added to the atelocollagen colloid used in the step (a).

In the meantime, the collagen-based matrix according to one embodiment of the present invention is prepared by the method for manufacturing a collagen-based matrix as described above.

### Advantageous Effects

According to the method for separating an atelocollagen, the process of ultrafiltration utilizing reusable filters and the process of diafiltration are combined and, thus by way of a single procedure line, atelocollagens can be extracted in a high purity from an animal tissue economically by removing impurities efficiently and conveniently.

In addition, according to the method for preparing a modified atelocollagen, the modified atelocollagen that is dissolved in a neutral solution, outstanding in the biocompatibility, being applicable for various formulations and more convenient for use, can be prepared. Advantageously, such a modified atelocollagen can be utilized intact for various fields including cosmetics, medicines and food etc. without any hydrolysis including peptidosis and enzymatic digestion of collagen.

Further, according to the method for manufacturing a collagen-based matrix, the collagen-based matrix is prepared by forming a bilayer collagen structure comprising a dense layer and a porous layer (2 collagen layers different from each other in the porosity), and crosslinking them in order to improve the mechanical strength of the collagen-based matrix. The 3-dimensional porous collagen-based matrix prepared by this process is advantageous to overcome the problem that a porous membrane is disrupted and lost partially in a solution, and detached from a dense layer easily.

### BRIEF DESCRIPTION OF DRAWINGS

The above and other objects, features and other advantages of the present invention will be more clearly understood to those skilled in this arts from the following detailed description taken in conjunction with the accompanying drawings.

FIG. 1 is a block diagram showing a purification apparatus (100) used to separate and purify an atelocollagen by the method for separating an atelocollagen according to the present invention that combines the process of ultrafiltration and the process of diafiltration.

FIG. 2 and FIG. 3 are a graph and a table, respectively showing a quantitative result of atelocollagens (0.5 mg/mL of sample concentration) purified by the method for separating an atelocollagen according to one embodiment of the present invention after removing water.

FIG. 4 is a photograph showing an electrophoretic result stained with Coomasie Brilliant Blue after performing a SDS-PAGE (sodium dodecyl sulfate polyacrylamide gel electrophoresis), and a graph showing a quantitative analysis of atelocollagens purified by the method for separating an atelocollagen according to one embodiment of the present invention.

FIG. 5 is a photograph showing collagen type 1 identified by performing an electrophoresis and western blotting with atelocollagens purified by the method for separating an atelocollagen according to one embodiment of the present invention.

FIG. 6 is a graph showing an analytic result of circular dichroism spectrum for atelocollagens purified by the method for separating an atelocollagen according to one embodiment of the present invention.

FIG. 7 and FIG. 8 are a graph and a table, respectively showing a quantitative result of pepsins in atelocollagens purified by the method for separating an atelocollagen according to one embodiment of the present invention, in order to identify whether pepsins used for removing telopeptides are left behind or not.

FIG. 9 is a data table showing a real-time PCR result for atelocollagens purified by the method for separating an atelocollagen according to one embodiment of the present invention, in order to identify whether HEV (Hepatitis E Virus) derived from a pig is detected or not.

FIG. 10 is a photograph showing an electrophoretic result of RT-PCR (Reverse Transcriptase PCR) for atelocollagens purified by the method for separating an atelocollagen according to one embodiment of the present invention, in order to identify whether JEV (Japanese Encephalitis Virus) derived from a pig is detected or not.

FIG. 11 is a photograph showing the solubility of the succinylated atelocollagen (ionized atelocollagen) obtained by the method for separating a modified atelocollagen according to one embodiment of the present invention, compared to that of conventional collagens.

### EXAMPLES

Practical and presently preferred embodiments of the present invention are illustrated more clearly as shown in the following examples. However, it should be appreciated that those skilled in the art, on consideration of this disclosure, may make modifications and improvements within the spirit and scope of the present invention. References cited in the specification are incorporated into the present invention.

### Example 1

### Separation of Atelocollaen

### Example 1-1

### Pretreatment of Animal Tissue

Above all, porcine skin was prepared as an animal tissue and washed with tap water. In this Example, porcine skin was utilized for an animal tissue, but various animal tissues containing collagen, for example cow tails, cartilage tissues, bone tissues, tendon tissues of pig and the like could be utilized naturally.

The porcine skin washed above was cut to pieces in 2 cm×10 cm of size. The resulting porcine skin was immersed in 0.1 to 1 M acetic acid solution and swollen at 4° C. for 16 to 24 minutes. From the porcine skin tissue swollen, lipids and epithelia were removed with a knife, and the resulting tissue was cut to pieces in 1 cm×1 cm of size after removing lipids and epithelia.

The resulting tissue cut to pieces in 1 cm×1 cm of size was washed 5 to 10 times with purified water, added in 90 to 99% ethanol solution, and stirred at 4° C. for 16 to 24 hours. Then, the tissue cut to pieces in 1 cm×1 cm of size was sieved to remove the ethanol solution, added again in 90 to 99% ethanol solution, and stirred at 4° C. for 5 hours or more.

Then, the resulting tissue treated by stirring in the ethanol solution was sieved, immersed in 0.1 to 1 M acetic acid solution, and swollen for 20 to 60 minutes. The swollen tissue was blended with 0.1 to 1 M acetic acid solution. This tissue blended was homogenized with a homogenizer.

### Example 1-2

### Removal of Telopeptide and Extraction of Atelocollaen

(1) The resultant solution obtained by performing the process of pretreatment in Example 1-1 was treated with pepsin, stirred at 4° C. for 24 to 72 hours, and then adjusted to pH 8 to inactivate pepsins.

(2) The resulting solution obtained by the step (1) was centrifuged at 7,000 to 15,000 g for 10 to 30 minutes at 4° C. Then, lipids in the upper layer and impurities in the lower layer were removed and solution from the middle layer was collected.

(3) The middle-layer solution separated by centrifuging was weighed, then added in 1 to 10 M NaCl solution, and stirred at 4° C. for 10 to 60 minutes so as to extract atelocollagens.

(4) The resulting solution processed in the step (3) was centrifuged so as to obtain an atelocollagen precipitate extracted.

(5) The atelocollagen precipitate extracted in the step (4) was added in 90 to 99% ethanol solution and stirred at 4° C. for 16 to 24 hours.

(6) The resulting solution processed in the step (5) was centrifuged again so as to obtain an atelocollagen precipitate, and the step (5) was repeated once more.

### Example 1-3

### Separation and Purification of Atelocollaen

(1) The resultant solution obtained by performing the extraction of atelocollagens in Example 1-2 was centrifuged to collect a precipitate, then added in 0.01 to 0.1 M urea solution and stirred at 4° C. for 16 to 24 hours.

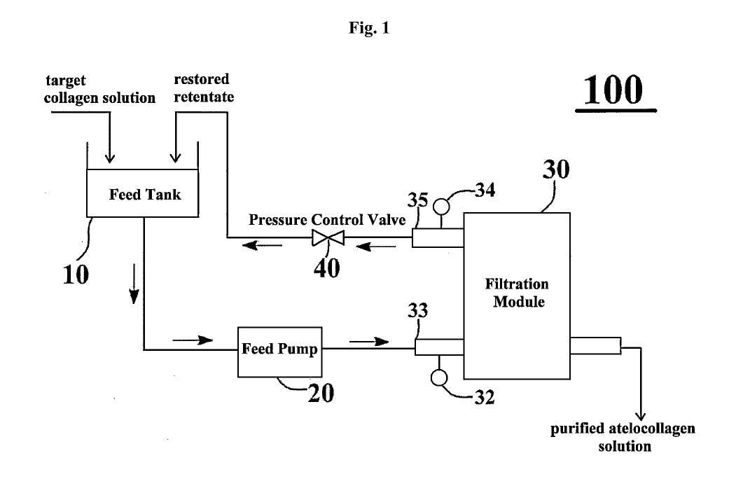

(2) The resulting solution obtained by the step (1) (hereinafter, referred to as “target collagen solution”) was purified by way of a single procedure line by conducting a method combining processes of ultrafiltration and diafiltration. For this purpose, a purification apparatus (100) was prepared as illustrated in FIG. 1. The apparatus of FIG. 1 can be manufactured manually, and assembled by using a commercial apparatus purchased from Pall Corporation (Model name: CENTRASETTE™ System).

(3) A filter membrane (not shown) was equipped within a filtration module (30) in the purification apparatus (100) of FIG. 1. NaOH remained on the filter membrane was washed out with purified water.

(4) A feed pressure gauge (32) and a retentate pressure gauge (34) installed in the filtration module of the purification apparatus (100) illustrated in FIG. 1 were observed, and all pressures on a feed portion (33) injecting “target collagen solution” and a retention portion (35) flowing out retentate left in the filtration module to restore into a feed tank (10) were released. The front side of a filter membrane was washed with water.

(5) A pressure control valve (40) in the purification apparatus (100) illustrated in FIG. 1 was adjusted to apply 10 to 30 psi of pressure on the retention portion (35) of the filtration module (30). Purified water injected freely from the feed portion (33) was pressurized to wash the rear side of the filter membrane. After washing, the pressure on the retentate portion (35) was released.

(6) A container housing “target collagen solution” and a container housing a purified atelocollagen solution were prepared and these containers were connected to the purification apparatus (100) of FIG. 1 via a hose.

(7) The pressure control valve (40) in the purification apparatus (100) of FIG. 1 was adjusted to apply 10 to 30 psi of pressure on the retention portion (35) of the filtration module (30). In this case, the “target collagen solution” flowing into the filtration module (30) through the feed portion (33) from the feed tank (10) was pressurized by pumping with a feed pump (20), the atelocollagens being passed through and filtrated over a filter membrane. As a result, the “target collagen solution” was ultrafiltrated primarily, and a part of atelocollagens purified were released from the filtration module (30) and collected in a container prepared previously.

(8) Then, in the ultrafiltration of the step (7), this filtration was stopped when the rate of a filtrated solution decreasingly reached approximately 1 g/min or less.

(9) Besides, in order to improve the convenience and efficiency of the process for separating an atelocollagen, a single procedure combining an ultrafiltration and a diafiltration by using the purification apparatus (100) illustrated in FIG. 1 was performed to increase the productive yield and the purity of atelocollagen. That is to say, the retentate excluded over a filter membrane in the filtration module (30) of the purification apparatus (100) illustrated in FIG. 1 was restored uniformly by way of the retention portion (35) of the filtration module (30) at a flow rate. In the step (8), when the ultrafiltration rate reached a uniform rate or less, the ultrafiltration was stopped and followed by the diafiltration for the retentate restored to the feed tank (10). Then, the retentate restored to the feed tank (10) was added in the same volume of purified water as the solution volume filtrated by the ultrafiltration, and diafiltrated 5 times or more by using the purification apparatus (100) illustrated in FIG. 1.

(10) The atelocollagen solution obtained by the ultrafiltration in the step (7) and the atelocollagen solution obtained by the diafiltration in the step (9) were adjusted to pH 7.0, and then lyophilized to obtain atelocollagens in a spongeform finally.

The method for separating an atelocollagen described in this Example has overcome the problems as follows. When several filters are overlapped, the purification could cost high and become inconvenient, because of consuming expensive filters a lot. That is to say, atelocollagens are partially purified by the ultrafiltration with reusable filters, until the ultrafiltration efficiency decreases below the predetermined filtration rate. Right after, the filtration module (30) can be applied for a diafiltration apparatus to filtrate retentates without replacing filters and retooling the apparatus. In other words, the method for separating an atelocollagen described in this Example can remove impurities efficiently and conveniently by way of a circulated single procedure line, extract and separate highly purified atelocollagens economically in a good yield from an animal tissue.

### Example 2

### Analytic Tests of Purity, and Safety etc. in Atelocollaen Extracted and Purified in Example 1

### Example 2-1

### Analysis of Purity, Denatured Status, and Types etc. in Purified Atelocollaen

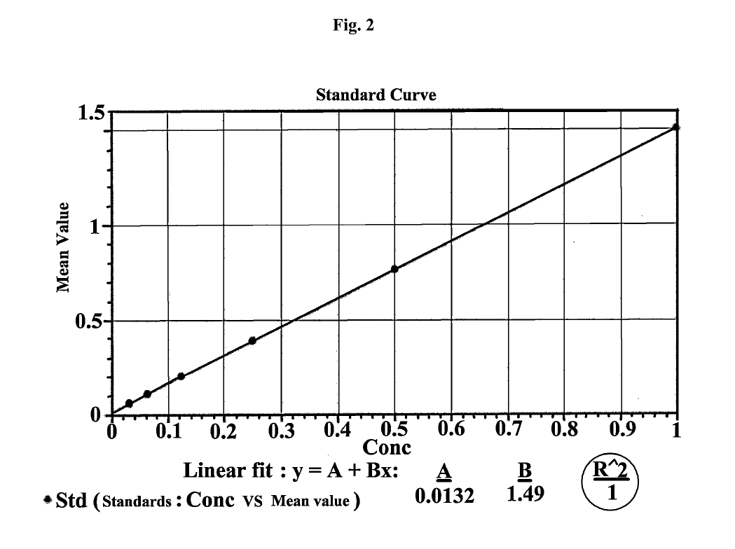

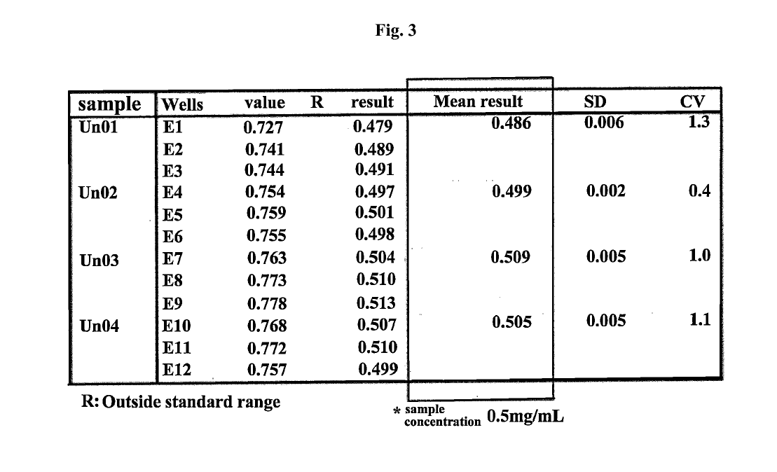

The atelocollagen extracted and purified in Example 1 (0.5 mg/mL of sample concentration) was quantitated after removing water. As a result, as illustrated in FIG. 2 and FIG. 3, the atelocollagen purified by the method for separating an atelocollagen according to one embodiment of the present invention was found to have 98% or more in the purity.

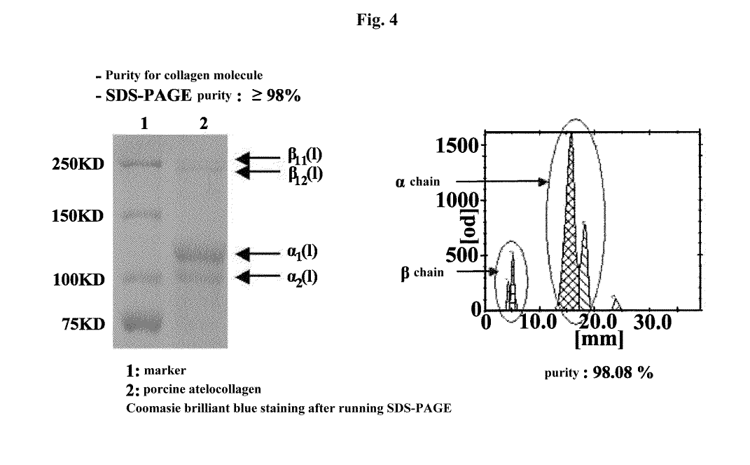

In addition, the atelocollagen extracted and purified in Example 1 was analyzed by performing a SDS-PAGE (sodium dodecyl sulfate polyacrylamide gel electrophoresis) and stained with Coomasie Brilliant Blue. As illustrated in FIG. 4, the atelocollagen purified by the method for separating an atelocollagen according to one embodiment of the present invention was found to have a peptide chain and to have 98% or more in its purity.

Besides, the atelocollagen extracted and purified in Example 1 was analyzed with a circular dichroism spectrum. As confirmed in the graph illustrated in FIG. 6, the atelocollagen purified by the method for separating an atelocollagen according to one embodiment of the present invention was observed not to be denatured and to maintain a 3-peptide helix (α chain) structure bound by hydrogen bonds.

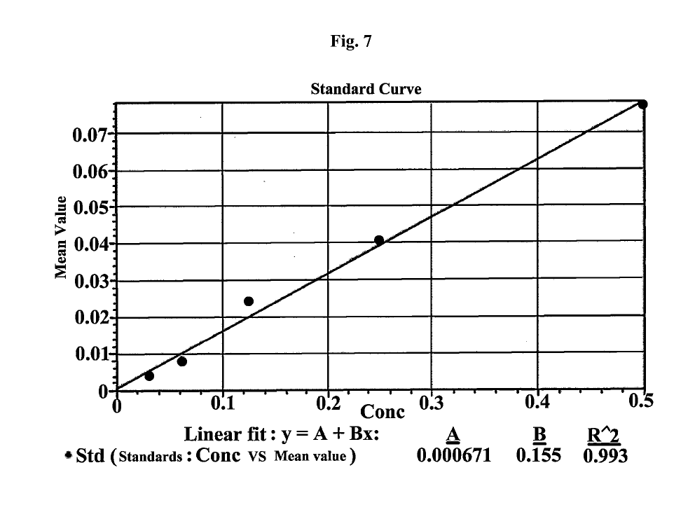

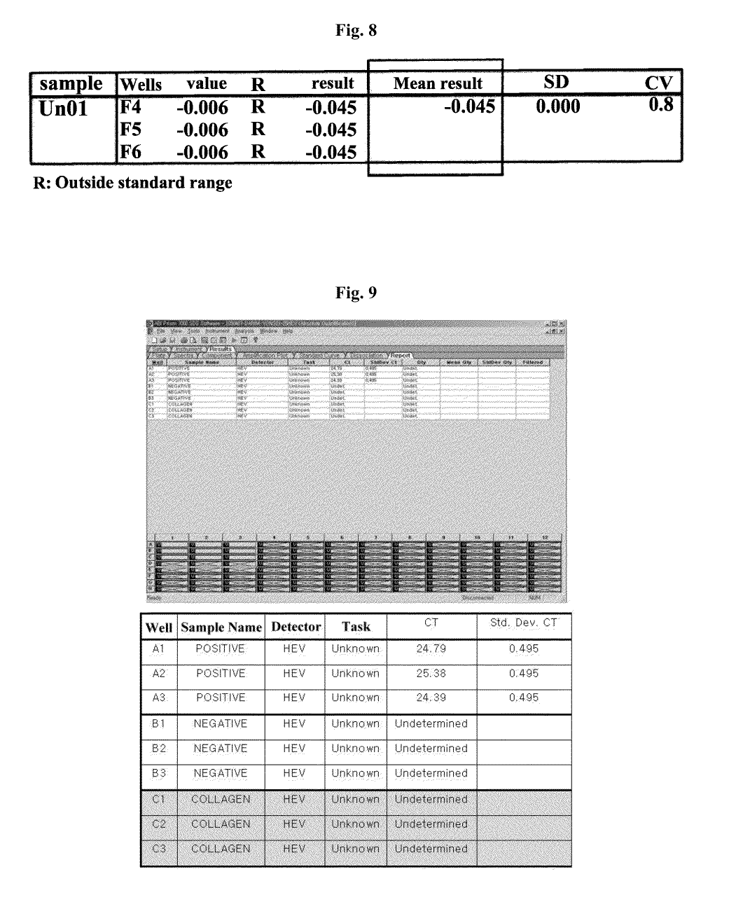

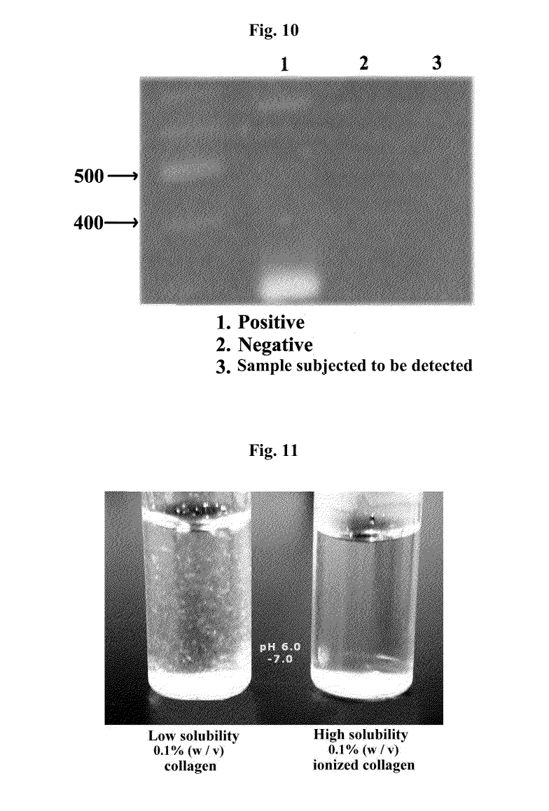

Furthermore, pepsins used for removing telopeptides were quantitated in order to identify whether the pepsin are left in the atelocollagen purified in Example 1. As a result, as illustrated in FIG. 7 and FIG. 8, it was observed that pepsins should not be detected in the atelocollagen purified by the method for separating an atelocollagen according to one embodiment of the present invention.

Therefore, the method for separating an atelocollagen according to the present invention is found effective to purify atelocollagens from an animal tissue in a high purity and without being denatured.

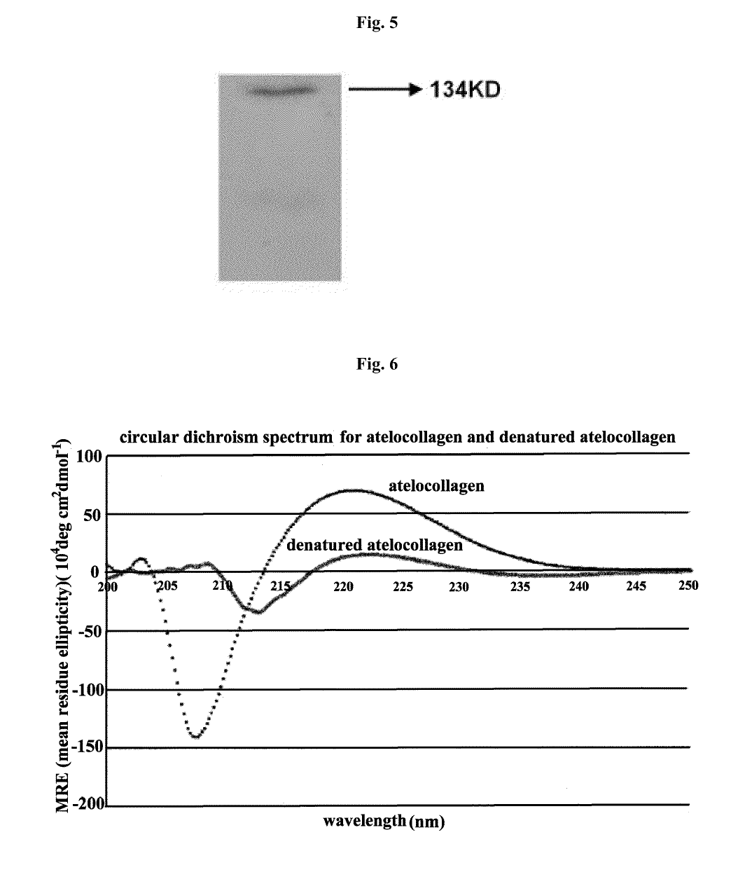

On the other hand, as illustrated in FIG. 5, the atelocollagen extracted and purified in Example 1 was analyzed by performing an electrophoresis and a western blotting so as to determine the collagen type. As a consequence, the atelocollagen purified above was identified as collagen type 1. In the western blotting, mouse anti-collagen type 1 monoclonal antibodies were utilized as a primary antibody, and rabbit anti-mouse IgGs conjugated with peroxidase were used as a secondary antibody.

### Example 2-2

### Safety Test of Purified Atelocollaen

Since the atelocollagen extracted and purified in Example 1 was derived from a porcine tissue, a safety test was conducted to detect a virus derived from a pig.

Above all, in order to identify whether HEV (Hepatitis E Virus) derived from a pig is detected or not in the atelocollagen extracted and purified in Example 1, a real-time PCR was performed. As a result, as illustrated in FIG. 9, the atelocollagen purified by the method for separating an atelocollagen according to one embodiment of the present invention was observed negative against HEV derived from a pig. This atelocollagen was identified harmless in a human, since HEV was not detected.

Then, in order to identify whether JEV (Japanese Encephalitis Virus) derived from a pig is detected in the atelocollagen purified in Example 1, an RT-PCR (Reverse Transcriptase PCR) was performed and electrophoresed. As confirmed in the photograph of electrophoretic results, it was observed that a gene band corresponding to JEV did not appear. Thus, the atelocollagen purified by the method for separating an atelocollagen according to one embodiment of the present invention was found harmless in a human, since JEV was not detected.

According to analytic data of purity, safety and the like in the purified atelocollagen as described above, the atelocollagen purified by the method for separating an atelocollagen according to one embodiment of the present invention was ascertained as a collagen type 1 and to have a high purity because of removing pepsins and other impurities added during removing telopeptides, and to be safe against animal virus infections. Therefore, the atelocollagen purified by the method for separating an atelocollagen according to one embodiment of the present invention can be applied intact for various fields including cosmetics, medicines and food etc.

### Example 3

### Preparation of Modified Atelocollaen

Hereinafter, the method for preparing a succinylated atelocollagen and an esterified atelocollagen that are modified to become soluble in a neutral solution, applicable for various formulations and more convenient for use and improve the biocompatibility, will be described. The method for preparing modified atelocollagens according to the present invention is ameliorated to increase the productive yield and the purity, compared to conventional methods.

### Example 3-1

### Preparation of Succinylated Atelocollaen

The method for preparing a succinylated atelocollagen according to one embodiment of the present invention is described as follows.

(1) Atelocollagen (purified one in Example 1 or commercial one, both are available) corresponding to 0.002 to 0.01 weight % was added in 0.1 M acetic acid solution, and stirred at 4° C. for 1 to 2 days to dissolve atelocollagens.

(2) Succinic anhydride was added to the resulting atelocollagen solution obtained in the step (1) in a ratio of 0.8 to 1.3 g per 1 g of atelocollagen added in the step (1), and maintained at around pH 9 to 10 for 10 minutes by using 0.05 to 1 M NaOH.

(3) The resulting solution obtained in the step (2) was stirred at 4° C. for 30 minutes.

(4) The resulting solution stirred in the step (3) was maintained at about pH 9 to 10 for 10 minutes by using 0.05 to 1 M NaOH.

(5) The resulting solution obtained in the step (4) was stirred at 4° C. for 30 minutes.

(6) The resulting solution stirred in the step (5) was maintained at around pH 9 to 10 minutes by using 0.05 to 1 M NaOH.

(7) The resulting solution obtained in the step (6) was stirred at 4° C. for 20 minutes.

(8) The resulting solution stirred in the step (7) was maintained at around pH 9 to 10 for 10 minutes by using 0.05 to 1 M NaOH.

(9) The resulting solution obtained in the step (8) was stirred at 4° C. for 10 minutes.

(10) The resulting solution stirred in the step (9) was adjusted at around pH 9 to 10 by using 0.05 to 1 M NaOH.

(11) The resulting solution obtained in the step (10) was adjusted at pH 4.03 by using 3 to 7 M HCl to precipitate a succinylated atelocollagen and stirred at 4° C. for 15 minutes.

(12) The resulting solution stirred in the step (11) was centrifuged to collect a precipitate of succinylated atelocollagen.

(13) The precipitate of atelocollagen obtained in the step (12) was added in distilled water adjusted to pH 4.03 by using 3 to 7 M HCl in a ratio of 20 mL per 1 g of the atelocollagen added in the step (1), and stirred at 4° C. for 15 minutes to wash.

(14) The resulting solution obtained in the step (13) was centrifuged to collect the washed precipitate of succinylated atelocollagen.

(15) The step (13) and the step (14) were repeated once more and the washed resulting precipitate of succinylated atelocollagen was lyophilized at −70° C. for 30 hours to obtain a succinylated atelocollagen finally.

The succinylation of atelocollagen prepared by the above-mentioned procedure is illustrated in Reaction Formula 1 as follows.

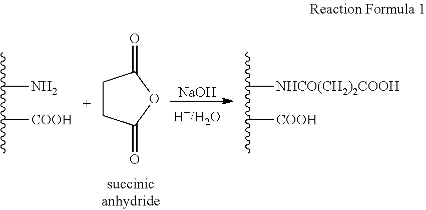

In the meantime, in case that the succinylated collagen is prepared by conventional methods, succinic anhydride is problematically insoluble at too high pH or at too low pH. Succinic anhydride can be dissolved most preferably at around pH 9 to 10, but not dissolved at pH 11 or more. In such a problem, the present inventors have found that when pH being changed according to the reaction of atelocollagen and succinic anhydride, the reaction velocity is lowered due to the decrease of solubility of succinic anhydride to decrease the productive yield. In order to settle the problem, an additional step of adjusting pHs repeatedly to 9 to 10 in the reacting solutions (the step (3) to the step (11)) was newly introduced.

That is to say, in the method for preparing a succinylated atelocollagen according to one embodiment of the present invention as described above, atelocollagen was reacted with succinic anhydride at a low temperature with being stirred for a predetermined period, and then adjusted to pH 9 to 10 for a predetermined period to promote the succinylation by dissolving succinic anhydride sufficiently. Indeed, the method for preparing a succinylated atelocollagen according to the one embodiment of the present invention improves the productive yield of succinylated atelocollagens.

### Example 3-2

### Preparation of Esterified Atelocollaen

The method for preparing an esterified atelocollagen according to one embodiment of the present invention is described as follows.

(1) Atelocollagen (purified one in Example 1 or commercial one, both are available) corresponding to 1 to 5 weight % was added in 70 to 90% ethanol (or methanol) to make a colloid solution, adjusted to pH 2 to 4 by adding 0.5 to 1 M acetic acid or 0.1 to 0.5 M HCl, and then stirred at 4° C. for 4 to 10 days.

(2) The atelocollagen colloid obtained in the step (1) was adjusted to pH 7.4 by using 0.1 to 0.5 M NaOH, and then centrifuged to collect a precipitate exclusively.

(3) The resulting precipitate obtained in the step (2) was added in purified water in a ratio of 10 to 100 mL per 1 g of atelocollagen precipitate, and then transferred into a dialysis bag to dialyze in a dialysis buffer.

(4) After stirring for 16 to 24 hours, the dialysis buffer was exchanged, and then exchanged repeatedly 3 to 12 times every 3 to 5 hours.

(5) The esterified atelocollagen precipitate dialyzed by the step (3) and the step (4) was lyophilized at −70° C. for 30 hours or more to obtain a lyophilized esterified atelocollagen.

The esterification of atelocollagen prepared by the above-mentioned procedure is illustrated in Reaction Formula 2 as follows.

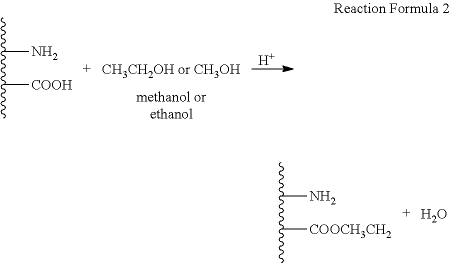

In typical methods for preparing an esterified atelocollagen, purified water is used simply for conducting a dialysis in order to increase the yield and the purity. In contrast, in the method for preparing an esterified atelocollagen according to one embodiment of the present invention, atelocollagens are blended in ethanol or methanol and the resulting atelocollagen colloid is neutralized, centrifuged to collect a precipitate exclusively, and then dialyzed by using a dialysis membrane so as to improve the purity and the yield.

### Example 4

### Evaluation of Physical Property in Modified Atelocollaen

The solubility of succinylated atelocollagen (anionic atelocollagen) obtained in Example 3 by the method for preparing a modified atelocollagen according to one embodiment of the present invention, was compared with that of typical atelocollagens. As confirmed in the photograph of FIG. 11, it was observed that the succinylated atelocollagen prepared by the method for preparing a modified atelocollagen according to the present invention has the high solubility at a neutral pH (pH 6.0 to 7.0).

As a consequence, the succinylated atelocollagen of binding atelocollagen with succinic anhydride obtained by the method for preparing a modified atelocollagen according to the present invention can dissolve even at a neutral pH due to its anionic property and improve the cell attachment, proliferation and migration. The esterified atelocollagen of binding atelocollagen with ethanol (or methanol) obtained by the method for preparing a modified atelocollagen according to the present invention is also advantageous to dissolve at a neutral pH, and can bind onto cells more rapidly and easily due to its anionic property.

Hence, according to the present invention, highly purified atelocollagens obtained by the ultrafiltration and the diafiltration are succinylated or esterified to be modified to dissolve even at a neutral pH. It is advantageous to be applied intact for various fields including cosmetics, medicines and food etc. without any hydrolysis including peptidosis and enzymatic digestion of collagens.

### Example 5

### Manufacturing of Collagen-Based Matrix in 3-Dimensional Matrix Structure

Hereinafter, the method for manufacturing a collagen-based matrix having a 3-dimensional matrix structure, wherein a dense layer and a porous layer (2 collagen layers different from each other in the porosity) are constructed by using atelocollagens and/or modified atelocollagens, and crosslinked to increase the mechanical strength as described in the present invention, will be demonstrated clearly according to the following steps.

The collagen-based matrix belonging to the present invention is composed of a bilayer of a dense layer and a porous layer, and basically the dense layer and the porous layer are prepared from the atelocollagen colloid.

### Example 5-1

### Preparation of Atelocollagen Colloid

Above all, in order to prepare an atelocollagen colloid for a porous membrane, the atelocollagen obtained in Example 1 was added in distilled water to correspond to 1 to 5 weight%, stirred and spread at 4° C. for 1 to 2 days, and then adjusted to pH 7.4 by adding 0.05 to 1 N NaOH.

In addition, in order to prepare an atelocollagen colloid for a dense membrane, the atelocollagen obtained in Example 1 was added in distilled water to correspond to 2 to 5 weight%, maintained, stirred and spread at 4° C. for 1 to 2 days, and then adjusted to pH 7.4 by adding 0.05 to 1 N NaOH.

### Example 5-2

### Manufacturing of Atelocollagen-Based Matrix

Hereinafter, an example for manufacturing a collagen-based matrix by using the atelocollagen colloid for porous membrane and the atelocollagen colloid for porous membrane is described as follows.

(1) Above all, the atelocollagen colloid for porous membrane prepared in Example 5-1 was spread on a petri dish or releasable plate to make a uniform membrane in the range of 0.05 to 1 mm, and then lyophilized within a lyophilizer at −60 to −80° C. for 1 to 2 days to prepare a porous membrane.

(2) In addition, the atelocollagen colloid for dense membrane prepared in Example 5-1 was spread on a releasable plate, and pressurized by 1 to 20 psi of pressure so as to make a uniform membrane in the range of 0.05 to 1 mm.

(3) Then, the dense membrane obtained in the step (2) was dried at a room temperature for 10 to 20 minutes, being softly layered onto the porous membrane obtained in the step (1), and air-dried at a room temperature for 1 to 2 days to obtain a bilayer by binding the dense membrane with the porous membrane.

(4) EDC was added to 90 to 99 weight % of ethanol with 10 to 100 mM of concentration, and stirred at 4° C. for 10 to 15 minutes to obtain a mixture. In the resulting mixture, the bilayer membrane obtained in the step (3) was immersed, and then crosslinked between the dense membrane and the porous membrane at 4° C. for 1 to 2 days. Otherwise, glutaraldehyde was added to 90 to 99 weight % of ethanol in a ratio of 0.5 to 1%, and stirred at 4° C. for 10 to 15 minutes to obtain a mixture. In the resulting mixture, the bilayer membrane obtained in the step (3) was immersed, and then crosslinked between the dense membrane and the porous membrane at 4° C. for 4 to 8 hours.

(5) The bilayer membrane crosslinked in the step (4) was washed 4 to 6 times by using distilled water in order to remove EDC or glutaraldehyde, and the washed resulting bilayer membrane was lyophilized at −60 to −80° C. for 1 to 2 days to manufacture a regular bilayer.

(6) The bilayer membrane prepared in the step (5) was tailored in 200 μm×200 μm×200 μm to 10 mm×15 mm×15 mm of size to complete the collagen-based matrix that increases the applicability for tissue-repair materials.

### Example 5-3

### Detailed Method for Manufacturing Atelocollagen-Based Matrix

Hereinafter, the method for manufacturing a collagen-based matrix described in the Example 5-2 is demonstrated more clearly as follows.

(1) Above all, the atelocollagen prepared in Example 5-1 corresponding to 2 weight % was added in distilled water, stirred and spread at 4° C. for 40 hours, to obtain a collagen colloid, and then adjusted to pH 7.4 by adding 0.5 N NaOH to obtain an atelocollagen colloid for preparing a porous membrane. In addition, the atelocollagen prepared in Example 5-1 corresponding to 4 wt % was added in distilled water, stirred and spread at 4° C. for 30 hours, to obtain a collagen colloid, and then adjusted to pH 7.4 by adding 0.5 N NaOH to obtain an atelocollagen colloid for a dense membrane.

(2) The atelocollagen colloid for preparing a porous membrane was spread on a releasable plate to form a uniform membrane in 0.05 mm of thickness, and then lyophilized within a lyophilizer at −70° C. for 30 hours to prepare a porous membrane. In addition, the atelocollagen colloid for dense membrane was spread on a releasable plate, and pressurized with a porous adsorption plate under 10 psi of pressure to form a uniform membrane in 0.5 mm of thickness.

(3) The dense membrane obtained in the step (2) was dried at a room temperature for 15 minutes, then being softly layered onto the porous membrane obtained in the step (2), and air-dried at a room temperature for 30 hours to obtain a bilayer membrane by binding the dense membrane with the porous membrane.

(4) EDC was added to 95 weight % of ethanol in 50 mM of concentration, and stirred at 4° C. for 15 minutes to obtain a mixture.

(5) In the resulting mixture of the step (4), the bilayer membrane obtained in the step (3) was immersed, and then crosslinked between the dense membrane and the porous membrane at 4° C. for 40 hours.

(6) In another method for crosslinking membranes, glutaraldehyde was added to 95 weight% of ethanol in a ratio of 0.625%, and stirred at 4° C. for 15 minutes to obtain a mixture.

(7) In the resulting mixture obtained in the step (6), the bilayer membrane obtained in the step (3) was immersed completely, and then crosslinked between the dense membrane and the porous membrane at 4° C. for 4 hours.

(8) The bilayer membranes crosslinked in the step (5) and the step (7) were washed for 15 minutes 5 times by using distilled water in order to remove EDC or glutaraldehyde.

(9) The washed resulting bilayer membranes were lyophilized at −70° C. for 30 hours to manufacture a regular bilayer.

(10) The bilayer membranes prepared in the step (9) were tailored in 200 μm×200 μm×200 μm to 10 mm×15 mm×15 mm of size to complete the collagen-based matrix that increases the applicability for tissue-repair materials.

On the other hand, in the method for manufacturing an atelocollagen-based matrix according to the present invention, the atelocollagen colloid can be replaced by a colloid mixture of atelocollagen and hyaluronic acid, which is being added by hyaluronic acid, a mucopolysaccharide capable of increasing cell migration when binding with collagen fibers. Furthermore, antibiotics such as penicillin etc. can be added to these colloids (See Korean Patent Publication No. 10-0947765).

Although the present invention has been illustrated and described with reference to the exemplified embodiments of the present invention, it should be understood that various changes, modifications and additions to the present invention can be made without departing from the spirit and scope of the present invention.

1. A method for separating an atelocollagen, which comprises steps of:
(a) preparing a sample containing atelocollagens without any telopeptide in a container;
(b) transferring the sample containing atelocollagens from the container toward a filtration module possessing a filter membrane, and performing an ultrafiltration by passing the sample through the filter membrane with applying a pressure on the filtration module to be filtrated;
(c) collecting an atelocollagen solution flowing out of the filtration module after filtrating over the filter membrane in the step (b);
(d) measuring a flow rate of the atelocollagen solution filtrated over the filter membrane to determine an ultrafiltration rate;
(e) stopping the ultrafiltration when the ultrafiltration rate reaches a predetermined level or less;
(f) collecting a retentate of the sample excluded on the filter membrane and restored from the container, adding water to the collected retentate, and then transferring it to the filtration module possessing a filter membrane to perform a diafiltration;
(g) collecting an atelocollagen solution flowing out of the filtration module after filtrating over the filter membrane in the step (f); and
(h) repeating the steps (f) and (g).

2. The method for separating an atelocollagen according to claim 1, wherein in the step (b), the sample containing atelocollagens is transferred from the container toward the filtration module possessing a filter membrane by pumping with a pump device, and 10 to 30 psi of pressure is applied on the filtration module.

3. The method for separating an atelocollagen according to claim 1, wherein in the step (e), the ultrafiltration is stopped when the ultrafiltration rate reaches 1 g/min or less.

4. The method for separating an atelocollagen according to claim 1, wherein in the step (f), the same volume of purified water as that of the solution filtrated by the ultrafiltration is added to the retentate restored to the container.

5. The method for separating an atelocollagen according to claim 1, wherein the diafiltration is repeated at least 5 times.

6. An atelocollagen prepared by the method as claimed in any one of claims 1 to 5.

7. A method for preparing a succinylated atelocollagen, which comprises steps of:
(a) reacting an atelocollagen solution with succinic anhydride, and maintaining the reaction solution of atelocollagen and succinic anhydride under a basic condition;
(b) stirring the reactant of atelocollagen and succinic anhydride for a predetermined period at a low temperature;
(c) maintaining the reactant of atelocollagen and succinic anhydride for a predetermined period at around pH 9 to 10 after stirring in the step (b);
(d) converting the reactant of atelocollagen and succinic anhydride to an acidic state by adding acids, and forming a precipitate of succinylated atelocollagen; and
(e) separating and obtaining the precipitate of succinylated atelocollagen.

8. The method for preparing a succinylated atelocollagen according to claim 7, wherein the step (b) and the step (c) are repeated at least 4 times.

9. The method for preparing a succinylated atelocollagen according to claim 7, which further comprises a step of washing the precipitate of succinylated atelocollagen with an acidic distilled water.

10. The method for preparing a succinylated atelocollagen according to claim 7, which further comprises a step of lyophilizing the precipitate of succinylated atelocollagen.

11. A succinylated atelocollagen prepared by the method as claimed in any one of claims 7 to 10.

12. A method for preparing an esterified atelocollagen, which comprises steps of:
(a) preparing an atelocollagen colloid by adding atelocollagens in ethanol or methanol, converting the atelocollagen colloid to an acidic state by adding acids, and then stirring it;
(b) converting the atelocollagen colloid stirred in the step (a) to a neutral state;
(c) collecting a precipitate of esterified atelocollagen by centrifuging the atelocollagen colloid converted to a neutral state in the step (b); and
(d) pouring the precipitate of esterified atelocollagen obtained in the step (c) into a dialysis membrane to perform a dialysis in purified water.

13. The method for preparing an esterified atelocollagen according to claim 12, which further comprises a step of lyophilizing the precipitate of esterified atelocollagen dialyzed in the step (d).

14. An esterified atelocollagen prepared by the method as claimed in claim 12 or 13.

15. A method for manufacturing a collagen-based matrix, which comprises steps of:
(a) spreading uniformly the atelocollagen colloid obtained by the method of claim 1 to form a membrane with a uniform thickness, and then lyophilizing to make a porous layer of collagen;
(b) spreading uniformly the atelocollagen colloid obtained by the method of claim 1, leaching out water and closely attaching collagen particles by pressing with a porous adsorption plate to form a dense layer of collagen;
(c) layering the porous layer of collagen formed in the step (a) onto the dense layer of collagen formed in the step (b), and air-drying them so as to primarily bind the porous layer of collagen to the dense layer of collagen; and
(d) generating crosslinking between the porous layer of collagen and the dense layer of collagen bound primarily in the step (c) by using a crosslinking means so as to secondarily bind the porous layer of collagen to the dense layer of collagen.

16. The method for manufacturing a collagen-based matrix according to claim 15, wherein the crosslinking means is EDC[1-ethyl-3-(3-dimethyl aminopropyl)carbodiimide] or glutaraldehyde.

17. The method for manufacturing a collagen-based matrix according to claim 15, which further comprises a step of washing out the crosslinking agent, when the crosslinking means is a crosslinking agent, and wherein a bilayer comprising the dense layer of collagen and the porous layer of collagen is further lyophilized, after washing out the crosslinking agent.

18. The method for manufacturing a collagen-based matrix according to claim 15, wherein hyaluronic acid is added to the atelocollagen colloid used in the step (a).

19. A collagen-based matrix prepared by the method as claimed in any one of claims 15 to 18.
